(19) United States
(12) Patent Application Publication
(10) Pub. No.: US 2014/0212338 A1
(43) Pub. Date: Jul. 31, 2014

(54) FRAGRANCE ENHANCER

(71) Applicant: Jeffrey Hopkins, N. Ridgeville, OH (US)

(72) Inventor: Jeffrey Hopkins, N. Ridgeville, OH (US)

(21) Appl. No.: 14/163,409

(22) Filed: Jan. 24, 2014

Related U.S. Application Data
(60) Provisional application No. 61/757,906, filed on Jan. 29, 2013.

(51) Int. Cl. A61L 9/03

(52) U.S. Cl. CPC A61L 9/032
USPC 422/124

(57) ABSTRACT

A fragrance enhancer and candle assembly has a candle and a holder for housing the candle. A body is mounted to an upper surface of the holder. A mounting bracket for holding a fan assembly is mounted to the holder. The fan assembly includes a plurality of inverted blades, and a gap is formed between the body and the holder to enable air flow through the body.

### CLAIM OF PRIORITY

This application claims priority from Provisional Patent Application Ser. No. 61/757,906; filed on Jan. 29, 2013 which is hereby incorporated by reference in its entirety.

### BACKGROUND OF THE DISCLOSURE

This disclosure relates to candles. More particularly, it relates to an apparatus for increasing the airflow from a lighted candle for faster circulation of the fragrance emitted from the candle.

Existing candles are often formed and sold within glass jars. While the candles often emit strong or aromatic fragrances when lit, the dispersement of the fragrance may be inhibited by a lack of airflow or the location of the candle in the room.

Thus, there is a need for an apparatus which improves the airflow and dispersement of a fragrance emitted from a candle.

### SUMMARY OF THE DISCLOSURE

The disclosure relates to candles. More particularly, it relates to an apparatus for increasing the airflow from a lighted candle to increase circulation and dispersement of a fragrance emitted from the candle.

In accordance with one aspect of the disclosure, a fragrance enhancer for a candle has a body which is configured to be mounted on an upper surface of a candle holder; a mounting bracket positioned at an upper end of the body; a fan assembly rotatably mounted to the mounting bracket; and the fan assembly has a plurality of inverted blades which disperse fragrance from the candle.

In accordance with another aspect of the disclosure, a fragrance enhancer and candle assembly has a candle and a holder for housing a candle; a body mounted to an upper surface of the holder; a mounting bracket for holding a fan assembly; wherein the fan assembly has a plurality of inverted blades; and a gap is formed between the body and the holder to enable air flow through the body.

Another aspect of the disclosure is an apparatus which is removably attached to an upper portion of a candle jar.

Yet another aspect of the disclosure is an apparatus which fits onto a conventional 14.5 ounce candle jar.

Another aspect of the disclosure is a fragrance enhancer which is made of aluminum to minimize the enhancer becoming overheated.

Still another aspect of the disclosure is air inlet holes in the apparatus to allow air into the candle to maintain a lit flame.

Yet another aspect of the disclosure is air inlet holes with different ornamental configurations.

Still another aspect of the disclosure is an inverted fan blade for more efficient airflow.

Another aspect of the disclosure is a series of angled blades which rotate and dispense scent from the candle.

Other aspects of the disclosure will become apparent upon a reading and understanding of the following detailed description.

### DESCRIPTION OF THE DRAWINGS

FIG. 1 is a perspective view of a candle jar with a fragrance enhancer in accordance with a preferred embodiment of the disclosure;

FIG. 2 is a top plan view of a fan mounting bracket of FIG. 1;

FIG. 3 is a top plan view of a fan of the fragrance enhancer of FIG. 1;

FIG. 4 is a side elevational view of the fan of FIG. 3;

FIG. 5 is a side elevational view in cross-section of a fragrance enhancer in accordance with another embodiment of the present disclosure;

FIG. 6A is a top plan view of a dispensing fan in accordance with another aspect of the disclosure;

FIG. 6B is a side elevational view of the fan of FIG. 6A;

FIG. 7A is a top plan view of a fan mounting bracket in accordance with another aspect of the disclosure;

FIG. 7B is a side elevational view of the fan bracket of FIG. 7A;

FIG. 8A is a top plan view of a lid in accordance with another aspect of the disclosure; and

FIG. 8B is a side elevational view of the lid of FIG. 8A.

### DETAILED DESCRIPTION OF THE DISCLOSURE

The disclosure relates to candles. More particularly, it relates to an apparatus for increasing the airflow from a lighted candle for faster circulation of the fragrance emitted from the candle.

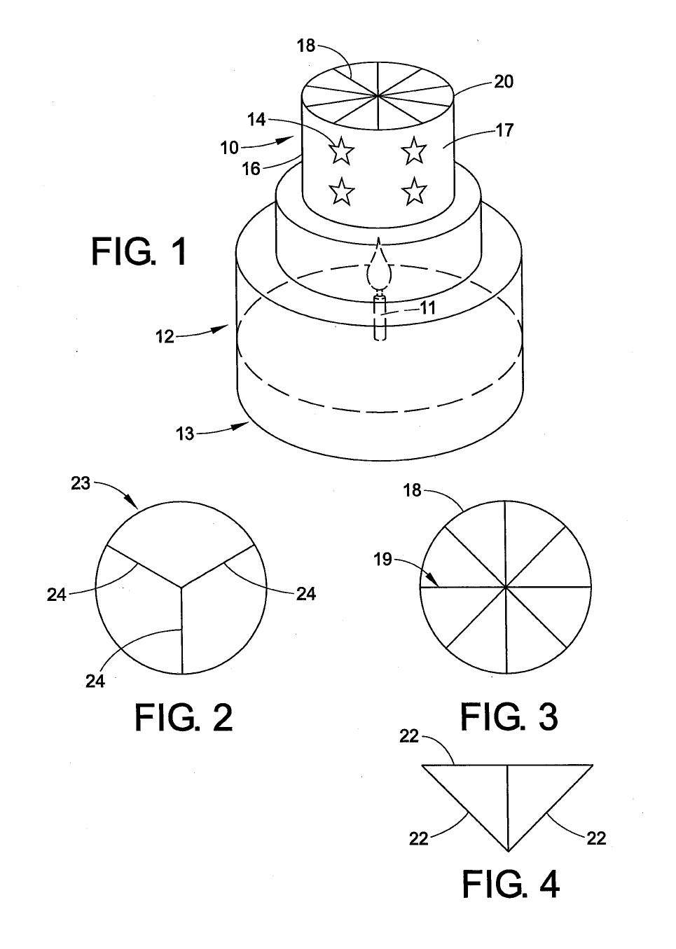

Referring now to FIGS. 1-4, a fragrance enhancer 10 in accordance with a first preferred embodiment is shown. The fragrance enhancer 10 is formed preferably from aluminum so as to prevent overheating of the enhancer from a lit wick 11 of a candle 12. The enhancer may be formed in a cylinder, cone or tapered configuration and is inserted or mounted to an upper end of a candle jar 13. The enhancer is preferably sized to fit on a conventional 14.5 ounce candle jar, but other sizes are contemplated by the disclosure. A plurality of air inlet holes 14 are formed on side walls 16 of the cone or cylinder 17 to allow air to enter the candle so the flame from the candle is not snuffed out. In accordance with one aspect of the disclosure, the air inlet holes can be stamped in different ornamental configurations such as Christmas trees, pumpkins for Halloween, and flags and stars for 4^{th} of July, etc.

Referring now to FIG. 3, a fan 18 with a blade assembly 19 is provided at an upper end 20 of the cylinder 17. Referring to FIG. 4, the fan preferably has inverted blades 22 for more efficient operation and improved air flow. Referring to FIG. 2, a mounting bracket assembly 23 has three spaced apart brackets 24 (more of these brackets can also be used) which are used to rotatably hold the fan arrangement and are positioned below the fan. The brackets 24 are mounted at upper end 20 of the cone. Heat from the lit candle wick rises and results in rotation of the fan blade, which in turn disperses the fragrance at a higher rate to the atmosphere outside of the candle jar.

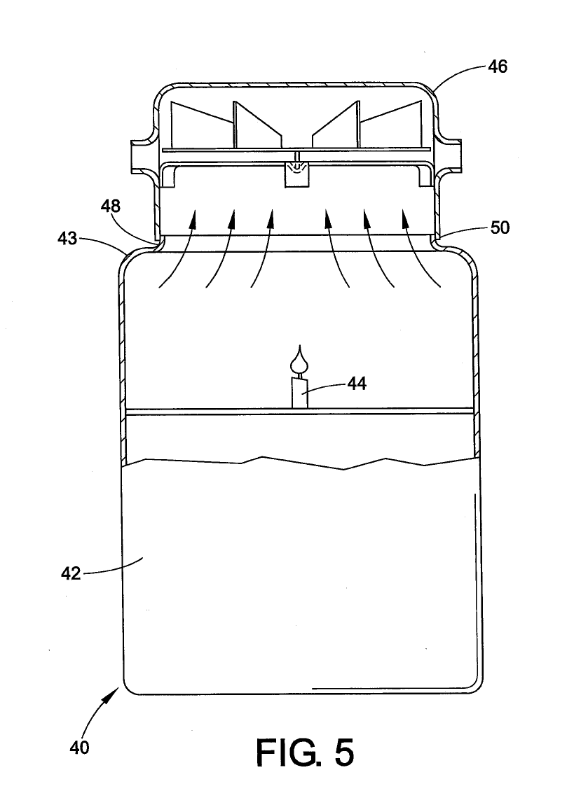

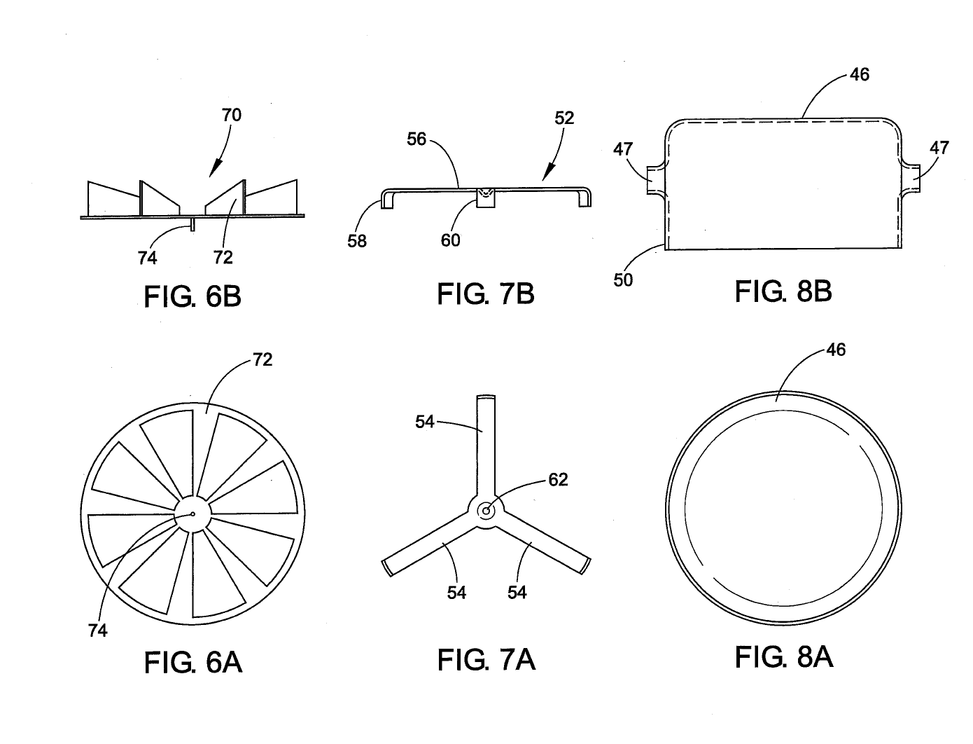

Referring now to FIGS. 5-7B, a fragrance enhancer 20 in accordance with another aspect of the disclosure is shown. A candle jar 40 has a candle 42 and wick 44 enclosed therein. A lid 46 is positioned on a top surface 43 of the candle jar. A space or gap 48 is formed near a bottom end 50 of the lid to allow cooler airflow to enter the lid. An fan bracket assembly 52 has three spaced apart brackets 54 having a top member 56 and a downwardly bent edge 58 which retains the bracket within the lid. A center member 60 has a hole or opening 62 therein for holding an inverted blade assembly 70. Blade assembly 70 has a plurality of spaced apart inverted blades 72 which disperse air and the fragrance from the candle. A pin or post 74 is used to mount the fan assembly to the bracket post 60. The cooler air flows through gap 48 and, along with the heat from the candle wick which arises, causes the fan blades to rotate, thus dispersing the fragrance into the atmosphere and through inlet holes 47.

The exemplary embodiment has been described with reference to the preferred embodiment. A motorized or mechanized fan assembly can also be used as part of the disclosure. Obviously, modifications and alterations will occur to others upon a reading and understanding of the preceding detailed description. It is intended that the exemplary embodiment be construed as including all such modifications and alterations insofar as they come within the scope of the above description or the equivalents thereof.

1. A fragrance enhancer for a candle, comprising:
a body which is configured to be mounted on an upper surface of an associated holder for said candle;
a mounting bracket positioned at an upper end of said body;
a fan assembly rotatably mounted to said mounting bracket;
wherein said fan assembly has a plurality of inverted blades which disperse fragrance from said candle.

2. The fragrance enhancer of claim 1, wherein said body comprises a cylinder.

3. The fragrance enhancer of claim 1, wherein said body comprises a cone.

4. The fragrance enhancer of claim 1, wherein said body comprises a plurality of air inlet holes.

5. The fragrance enhancer of claim 4, wherein said air inlet holes have an ornamental conformation.

6. The fragrance enhancer of claim 1, wherein said fan blades are rotated by heat emanating from said candle.

7. The fragrance enhancer of claim 1, wherein said mounting bracket comprises three spaced apart brackets.

8. A fragrance enhancer and candle assembly, comprising:
a candle and a holder for housing said candle;
a body mounted to an upper surface of said holder;
a mounting bracket for rotatably holding a fan assembly mounted to said body;
wherein said fan assembly comprises a plurality of inverted blades; and
a gap is formed between said body and said holder to enable air flow through said body.

9. The fragrance enhancer of claim 8, wherein said mounting bracket comprises three spaced apart brackets.

10. The fragrance enhancer of claim 9, wherein said brackets each has a top member and downwardly bent outer edges.

11. The fragrance enhancer of claim 10, wherein said mounting bracket comprises a center member having an opening for receiving said fan assembly.

12. The fragrance enhancer of claim 11, wherein said fan assembly comprises a post which is matingly received by said opening in said center member of said bracket.

13. The fragrance enhancer of claim 8, wherein said inverted blades are rotated by heat emanating from said candle.

14. The fragrance enhancer of claim 8, wherein said inverted blades are rotated by airflow through said gap.
